(19) United States
(12) Patent Application Publication
(10) Pub. No.: US 2023/0277217 A1
(43) Pub. Date: Sep. 7, 2023

(54) DEVICES HAVING A HANDLE PORTION, AN INTRAVAGINAL DILATOR PORTION, AND A VIBRATION MOTOR

(71) Applicants: North Carolina State University, Raleigh, NC (US); The University of North Carolina at Chapel Hill, Chapel Hill, NC (US)

(72) Inventors: Chandler Sizer, Wake Forest, NC (US); Alan Joseph Rosenbaum, Apex, NC (US); Abigail Scheer, Garner, NC (US)

(21) Appl. No.: 18/118,507

(22) Filed: Mar. 7, 2023

Related U.S. Application Data
(60) Provisional application No. 63/317,353, filed on Mar. 7, 2022.

(51) Int. Cl. A61B 17/42; A61M 29/00

(52) U.S. Cl. CPC A61B 17/42; A61M 29/00; A61M 2210/1475

(57) ABSTRACT

In some examples, a device includes a handle portion and an intravaginal dilator portion. The handle portion includes a curved handle portion; a battery within the handle portion; a motor housing including a vibration motor coupled to the battery; an activation button configured to activate the vibration motor; and a faceplate and one or more locating recesses within the faceplate. The intravaginal dilator portion includes a dilator plate and a motor housing recess configured to receive the motor housing when the intravaginal dilator portion is attached to the handle portion; and one or more locating pins configured to fit within the locating recesses of the handle portion when the intravaginal dilator portion is attached to the handle portion.

### CROSS-REFERENCE TO RELATED APPLICATIONS

This application claims benefit of U.S. Provisional Application Ser. No. 63/317,353, filed on Mar. 7, 2022, the disclosure of which is incorporated herein by reference in its entirety.

### TECHNICAL FIELD

The subject matter described herein relates to devices, systems, and methods of using devices having a handle portion, a dilator portion, and a vibration motor for treatment of dyspareunia and pelvic pain.

### BACKGROUND

By 2025, advances in cancer treatment will enable 15.1 million American and European female cancer survivors to live for decades beyond initial cancer diagnosis. Approximately half of these women will develop severe dyspareunia (pain with intercourse) because of their cancer treatment. Even with non-gynecologic malignancies, surgery, radiation, or chemotherapy can cause dyspareunia through alterations in vaginal anatomy, tissue dryness or atrophy, and reduced elasticity. Pelvic floor physical therapists use several different devices during in-clinic and at-home therapy to treat dyspareunia. Current devices target singular therapy needs: (i) vaginal dilators expand vaginal tissue, (ii) pelvic wands provide internal massage and myofascial release, and (iii) vibration devices reduce soreness and pain through relaxation. Consequently, female cancer survivors (who routinely employ multiple therapy methods) must purchase multiple devices to achieve therapy needs as well as to make adequate progression. Pelvic floor physical therapy is typically conducted 10-20 minutes a day, 3-5 times per week, and potentially for the remainder of a patient's life. This therapy can be painful and emotionally difficult, as it is often an unwanted reminder of their cancer battle.

### SUMMARY

In some examples, a device includes a handle portion and an intravaginal dilator portion. The handle portion includes a curved handle portion; a battery within the handle portion; a motor housing including a vibration motor coupled to the battery; an activation button configured to activate the vibration motor; and a faceplate and one or more locating recesses within the faceplate. The intravaginal dilator portion includes a dilator plate and a motor housing recess configured to receive the motor housing when the intravaginal dilator portion is attached to the handle portion; and one or more locating pins configured to fit within the locating recesses of the handle portion when the intravaginal dilator portion is attached to the handle portion.

In some examples, a system includes a handle portion, an intravaginal dilator portion, and a charging base. The handle portion comprises a battery within the handle portion; a motor housing including a vibration motor coupled to the battery; one or more handle charging contacts coupled to the battery; and an activation button configured to activate the vibration motor. The intravaginal dilator portion comprises a motor housing recess configured to receive the motor housing when the intravaginal dilator portion is attached to the handle portion. The charging base comprises one or more base electrical contacts configured to contact the handle charging contacts when the handle portion is attached to the charging base.

In some examples, a method includes pressing together handle portion of a device and an intravaginal dilator portion of the device and rotating until one or more locating pins of the intravaginal dilator fit within one or more locating recesses of the handle portion; further rotating to connect the handle portion of the device to the intravaginal dilator portion; and pressing an activation button on the handle portion of the device and activating a vibration motor within the handle portion of the device. The method can further include using the device for treatment of dyspareunia and pelvic pain, e.g., by performing pelvic floor physical therapy and/or by combining therapy modalities, such as vaginal dilation, trigger point massage, myofascial release, and vibration therapies.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described hereinbelow.

### BRIEF DESCRIPTION OF THE DRAWINGS

The subject matter described herein will now be explained with reference to the accompanying drawings of which:

FIGS. 1A-11 illustrate an example treatment device that can be used for treatment of dyspareunia and pelvic pain;

FIG. 2A is a cross-sectional view of the device in a configuration where the vibration motor is located in the dilator portion;

FIG. 2B is a side view of the device including a cross-sectional view of the dilator portion;

FIG. 3 is a side view of the handle portion of the device;

FIG. 4A is a cross-sectional view of the dilator portion of the device;

FIG. 4B is a cross-sectional view of the dilator portion in a configuration with one or more locator pins;

FIG. 5A is a cross-sectional view of the dilator portion of the device;

FIG. 5B is a cross-sectional view of the dilator portion in a configuration with one or more locator pins and where the dilator portion lacks the vibration motor;

FIG. 6 is a side view of the exterior of the dilator portion of the device;

FIG. 7 is a line-up view of a plurality of different dilator attachment portions of the device;

FIG. 8A is a block diagram of an example system for charging the handle portion of the device; and

FIG. 8B is a top view of an example configuration of the charging base.

### DETAILED DESCRIPTION

The disclosed subject matter may include various embodiments pertaining to a treatment device or related system for dyspareunia and pelvic pain. For example, some embodiments may be based upon a platform which couples a therapeutic-diagnostic device with a mobile application to enable complex, highly individualized dyspareunia management. In some embodiments, a treatment device may include one or more of the following features: vibration for stimulating blood flow and restoring lubricating glands, and vaginal dilation for stretching tissues, mobile app integration that allows user control, telemedicine capability for pelvic floor physical therapy providers to evaluate and adapt the plan of care, and body positive and self-care encouragement to address non-physical aspects of dyspareunia management and improve treatment adherence. These features may guide the patient to perform appropriate therapy during treatment sessions aimed at improving their unique causes of dyspareunia while collecting data that may be used by the clinician to monitor the efficacy of treatment and to diagnose acute changes or symptoms. Such symbiotic therapeutic-diagnostic integration can contribute to performance and create value for patients and providers alike.

In some embodiments, a treatment device (e.g., an intravaginal device) or related system in accordance with various aspects described herein may include one or more of the following features: an ergonomic handle allowing improved manipulation and ergonomics during device use, the capacity for combined vibration and dilation, a curvature to the dilator shaft which allows for pressure to be applied rotationally and laterally, and/or a tapered dilator tip for gradual insertion into the vaginal canal. For example, a treatment device may combine therapy modalities, such as vaginal dilation, trigger point massage, myofascial release, and vibration therapies.

As alluded to above, user adherence to pelvic floor physical therapy is a major challenge. In some embodiments, a treatment device in accordance with various aspects described herein and an associated medical application may utilize patient activation. For example, a treatment device and a related medical application can provide patient users the knowledge, skills, techniques, and confidence to manage their own health and healthcare to produce better health outcomes. As such, emotional, physical, and functional challenges cancer survivors face can be more effectively mitigated.

In some embodiments, a treatment device (e.g., an intravaginal device) or related system in accordance with various aspects described herein may enhance treatment by facilitating custom therapies such as vaginal dilation, tissue vibration, myofascial release, and trigger point massage. A feature of a treatment device in accordance with various aspects described herein is an ergonomically contoured handle that allows the device to be held in several different orientations as the patient user performs therapy in various positions. Notably, such functionality/configuration can eliminate strain placed on the wrist and shoulder during the 10-20 minute therapy sessions. For example, a patient user may comfortably grip and rotate the device throughout various positions, e.g., supine, hands and knees, and side-lying positions.

In some embodiments, a treatment device (e.g., an intravaginal device) or related system in accordance with various aspects described herein may reduce a patient's need to purchase multiple dilators as the size gradation is more efficient and conducive to effective dilation and internal massage requirements. For example, patients or users can avoid pelvic wand device and vibrator device purchases since the dilator form and function allows for appropriate myofascial release and internal massage techniques as well as vibration therapy. Further, a multi-function design in accordance with aspects described herein may dramatically improve storage and portability as well as product cleaning requirements and/or maintenance concerns.

In some embodiments, a device form and/or a charging mechanism described herein may also mitigate any visual stigma associated with sex toys. For example, women may prefer keeping a treatment device visible (and charging) to aid their prospective memory while serving as a visual reminder to conduct their therapy. In this example, in contrast to other intravaginal devices, a treatment device or related system in accordance with aspects described herein may be designed such that a user's reluctance to keep the device visible is reduced or mitigated, e.g., by improving aesthetics and/or reducing visual similarities to sex toys.

In some embodiments, a treatment device (e.g., an intravaginal device) or related system in accordance with various aspects described herein may include or utilize a corresponding mobile application that can communicatively connect with the treatment device via wireless means (e.g., Bluetooth, Wi-Fi, etc.). For example, a mobile application may provide therapy-related education (e.g., device usage instructions and/or related treatment information) and may also provide an exercise diary or related diary features to a user of a treatment device. In this example, the mobile application or its diary features may allow for better tracking of therapy frequency/duration, pain/complications, progress, and clinician communication. Through such diary features, patients can document any therapy concerns or goals to help improve therapy adherence and self-efficacy. Further, if permitted by a user, clinicians may receive (e.g., via the device-related mobile application) complete at-home therapy metrics and can use the data to optimally adjust treatments. In another example, physical therapists may be granted (e.g., by a user) permission to export the user's progress report and attach it to their electronic health record (EHR) account which can increase patient data collection and progress tracking.

In some embodiments, by providing multiple therapeutic functions in a treatment device or related system in accordance with various aspects described herein, market research suggests that such a device or system may increase access to for as many as 10-20% of women in the target population, e.g., by reducing the number of in-person pelvic floor physical therapy visits, improved device ergonomics, mobile application-based education, progress tracking, and personalized therapy. This research data suggests that in the United States and European Union an additional 4.3-8.6 million women could gain access to dyspareunia care. This is a sizable population that will receive meaningful quality of life improvement. This value proposition has been validated through numerous patient and provider interviews. The market currently serving women with dyspareunia fails to recognize that this population is highly heterogeneous, including women who suffer from vastly different conditions such as vaginal atrophy, endometriosis, vaginismus, painful bladder syndrome, irritable bowel syndrome, musculoskeletal disorders, recurrent vaginitis, intravaginal scar tissue, or are postpartum. At present, no current device targets cancer survivors despite being the most complicated use case due to their altered anatomy, tissue, comorbidities, and access-to-care. The design of the disclosed device specifically targets these users and will differentiate from other products that address pelvic floor population at-large. Long term, as the device targets the most complicated and challenging individuals, it has potential to improve the therapy experience for broader pelvic health audiences.

While a vast majority (e.g., 87%) of female cancer survivors in a survey viewed sexual function as important to their quality of life, 40% reported chronic pelvic pain and 50% reported significant dyspareunia due to cancer treatments that negatively impacts their relationships, body image, and self-esteem. Sexual health conversations are deprioritized in oncology follow-up visits as 33% of gynecological cancer patients reported they did not have any sexual health discussions with their clinician. Additionally, pelvic floor physical therapists typically only practice in major metropolitan areas and these practices often have month long waitlists. These barriers delay or prohibit treatment and can allow the conditions to reach a debilitating stage. This may prevent women from having acceptable sexual function for the remainder of their lives. The design of this system addresses a silent problem that millions of female cancer survivors are battling. The disclosed device/system improves patient's self-advocacy, their ability to set goals, and reinforces sexual health as a priority. Through a user-centered design lens, the device is pushing forward the women's health med-tech space. It is designed for women, by women. From the voices of female cancer survivors, we empathize with the emotional and functional qualities their pelvic floor therapy tool carries—it is an artifact of an emotional cancer battle, loss of self, and hope for a return to normalcy. The aesthetic and functional design is driving users' wants and needs to the forefront of a device that they may carry with them the rest of their lives.

During at-home therapy, women may be tasked with laying in various positions that simulates sexual intercourse. The high variability in these positions can create wrist and shoulder strain throughout a 20-minute session. Women must also be able to adjust and rotate their device to the 12, 3, 6, and 9 o'clock positions (e.g., 0, 90, 180, and 270 degree positions) to accurately massage and target their various pelvic floor muscles and/or vaginal tissue. In some embodiments, a treatment device (e.g., an intravaginal device) or related system in accordance with various aspects described herein may include a handle form that allows women to grip the device at a distal and proximal position. The handle form may allow women the freedom to determine their desired leverage during internal massage. For example, various grip positions may provide users with the ability to rotate and manipulate the intravaginal component with ease and comfort.

In some embodiments, a device handle of a treatment device or related system in accordance with various aspects described herein may include an overmolded grip (e.g., a thermoplastic polyurethane (TPU), a liquid silicone rubber (LSR), or a rubber or rubber-like overmolded grip) that provides additional friction for grip when users may have traces of lubrication on their hands. In such embodiments, the device handle design may be sensitive to the weight dynamic between the handle and intravaginal components, e.g., an 80-20, 75-25, or 63-37 handle-to-intravaginal components weight ratio. For example, a majority (e.g., greater than 50 percent) of the weight of the treatment device may reside in the handle so as to not cause additional strain in the wrist with extra mass in the dilator.

In some embodiments, e.g., using a two-stage overmold, the weight of each attachable dilator may be reduced through an insert component and thin silicone overmold. This reduction in weight may be especially apparent in the largest sizes in contrast to a solid silicone or a silicone core design that would shift the weight toward the end of the device's fulcrum point. In some embodiments, an insert component of a dilator may ensure that the rigidity of the smaller dilator sizes can withstand the pressure and torque that users may exert during internal massage.

In some embodiments, the size, curve, and form of various dilators described herein may have been meticulously vetted with both patients and physical therapists. Further, the extended length and curve of various dilators described herein may ensure that women can reach specific trigger points and muscles for trigger point massage and myofascial release, thereby eliminating the need to purchase additional pelvic wands. In some embodiments, e.g., where a treatment device or dilators thereof are covered in medical grade silicone material, the medical grade silicone material may be durable, soft touch, and appropriate for intravaginal use and daily cleaning with soap and water. In some embodiments, a range of sizes for various dilators described herein may eliminate the frequency of change sizes but will accommodate 95% of the oncology dyspareunia patient population.

Reference will now be made in detail to various embodiments of the subject matter described herein, examples of which are illustrated in the accompanying drawings. Wherever possible, the same reference numbers will be used throughout the drawings to refer to the same or like parts.

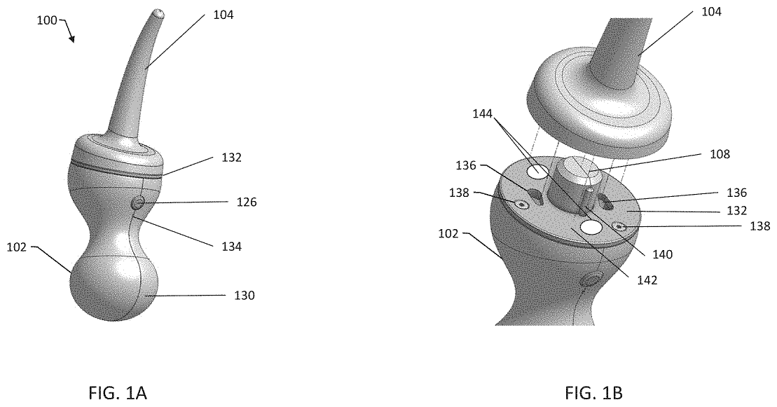

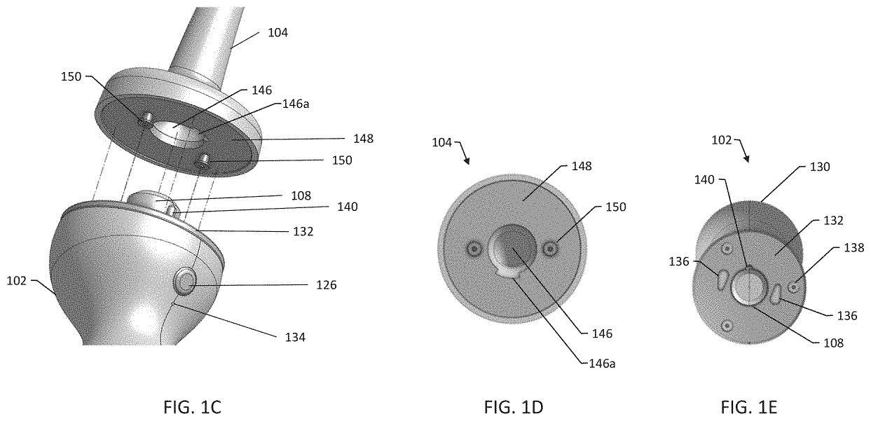

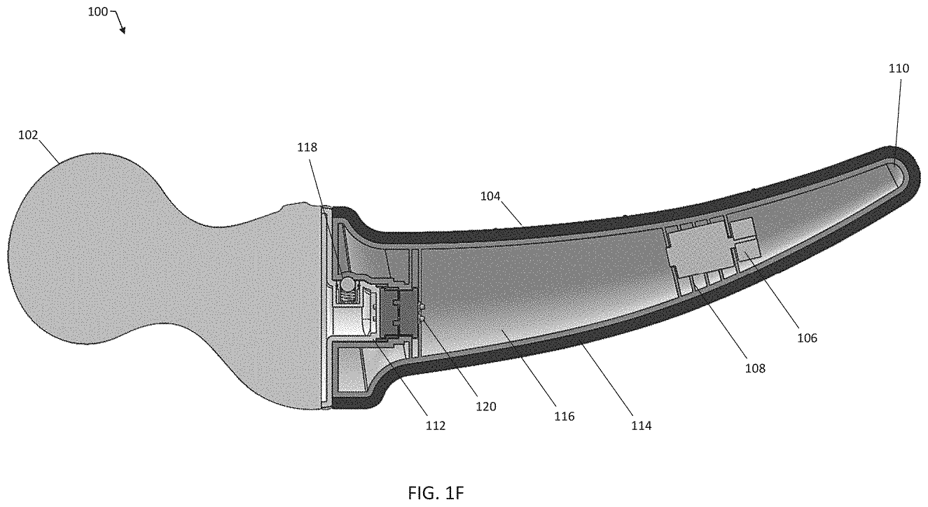

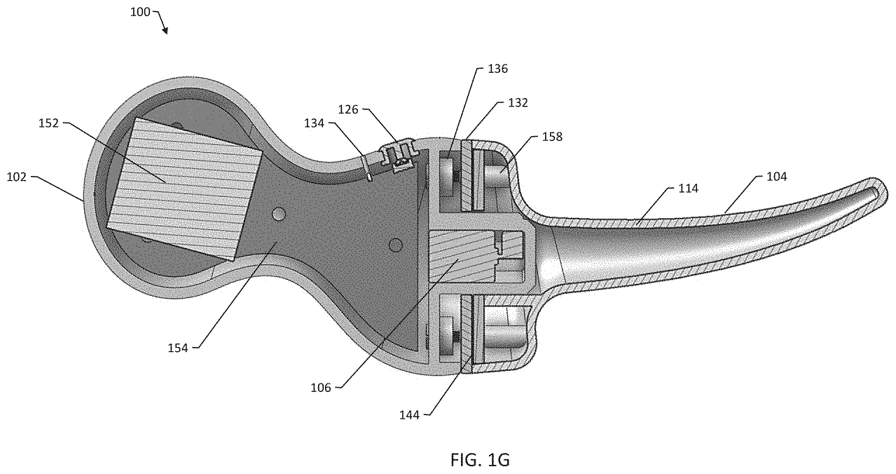

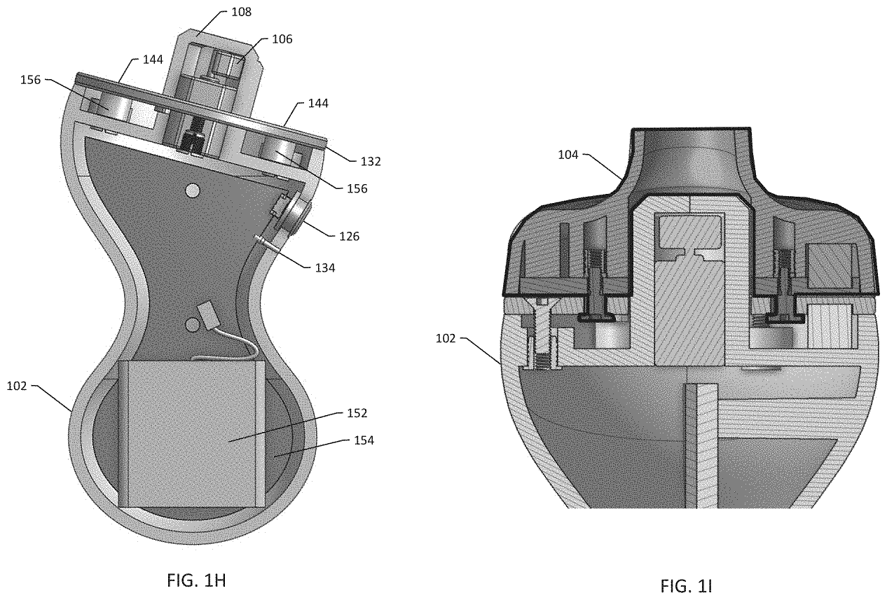

FIGS. 1A-11 illustrate an example treatment device 100 that can be used for treatment of dyspareunia and pelvic pain. The device 100 includes a handle portion 102 and an intravaginal dilator portion 104. The dilator portion 104 attaches to the handle portion 102, and then a user holds the handle portion 102 with a hand while using the dilator portion 104 to perform therapy, e.g., pelvic floor physical therapy. The device 100 assists patients with achieving pelvic floor muscle relaxation through vibration, vaginal dilation, myofascial release (deep tissue massage), and trigger point massage (internal muscle knots).

FIG. 1A shows on overview of the device 100 in an assembled state, i.e., with the dilator portion 104 attached to the handle portion 102. The handle portion 102 includes a curved handle portion 130 sized to fit comfortably in a human hand. The handle portion 102 can be formed, e.g., from a globe and a partial globe that are off-set from each other which are fused with a smooth transition between them, so that the curved handle portion 130 can be generally spherical. The shape, size, and design of the handle portion 102 are configured for ergonomics so that a user can comfortably grasp the handle portion 102 while performing therapy, e.g., pelvic floor physical therapy.

The handle portion 102 includes an activation button 126, a faceplate 132, and an indicator light 134. The activation button 126 can be configured to active and deactivate the device 100 and, in some examples, to cause the device to cycle between operational modes. In some embodiments, the activation button 126 is positioned on the top of the partial globe has the faceplate 132 and attaches to the dilator portion 104. The size and location of activation button 126 can be configured so that it does not interfere with common ways that users would want to hold the handle during therapy. For example, the activation button 126 may be designed to not be inadvertently activated/deactivated but still located such that a user can press it when needed. Notably, in some embodiments, the activation button 126 is configured to cause the device to vibrate and/or cycle through different vibration speeds.

In some examples, the activation button 126 is a momentary push-button, configured as an input device to send a signal to a processor or microcontroller every time it is pressed. Physically, a momentary push-button is usually circular or square in shape, with a small button or plunger in the center that can be depressed when pressed. The button may be made of plastic, metal, or other materials and may have a small tactile feedback or click when pressed, to give the user a physical confirmation that the button press has been registered.

A momentary push-button typically has two electrical contacts, which are normally open (i.e., not connected) when the button is not pressed. When the button is pressed, the contacts are momentarily closed, allowing current to flow and sending a signal to the processor or microcontroller. In some cases, momentary push-buttons may be configured with additional features such as a light emitting diode (LED) to provide visual feedback, or with a protective cover or guard to prevent accidental pressing. The activation button 126 may be mounted using, e.g., mounting screws or snap-in clips.

The faceplate 132 can be made of any suitable material, e.g., plastic or a metal such as stainless steel. The faceplate 132 serves as an interface between the dilator portion 104 and the handle portion 102. The indicator light 134, which is typically an LED, is a small light that is used to indicate the charging status of the device 100. The indicator light 134 serves as a visual cue to let the user know whether the device is currently charging or fully charged.

Physically, the indicator light 134 can be a small, circular or rectangular light that is embedded into the device's casing. The indicator light 134 may be colored, with common colors including green, red, and amber. When the device 100 is connected to a power source and charging, the indicator light 134 will typically turn on and remain illuminated until the battery is fully charged. The color of the indicator light 134 may change as the battery charges, with some devices using a different color to indicate different levels of charge. For example, the indicator light 134 may be green when the battery is above a certain level, amber when the battery is low, and red when the battery is critically low and needs to be charged immediately.

FIG. 1B shows the device 100 in a separated state, i.e., with the dilator portion 104 separated from the handle portion 102. FIG. 1B shows that the handle portion 102 includes a motor housing 108 for a vibration motor and locating recesses 136 within the faceplate 132 configured for receiving dilator attachment pins in the dilator portion 104. The handle portion 102 includes fasteners 138 (e.g., screws) that hold the faceplate 132 to the housing of the handle portion 102.

The handle portion 102 includes a connecting tab 140 configured to prevent an incorrect orientation of the dilator portion 104 to the handle portion 102 when the two parts 102 and 104 are put together. The connecting tab 140 is a protrusion that sticks out from the faceplate 132 or the motor housing 108 or both, and the connecting tab 140 is sized and shaped to fit into a notch on the dilator portion 104. In the example shown in FIG. 1B, the connecting tab 140 is shaped as a portion of a cylinder that extends away from the faceplate 132 along the motor housing 108.

FIG. 1B also shows the location 142 of locating magnets. The magnets themselves are located underneath the faceplate 132; however, the location 142 shown in FIG. 1B is where magnetic force may be exerted to secure the connection between the handle portion 102 and the dilator 104.

FIG. 1B shows charging contacts 144 for electrically connecting the handle portion 102 to a charging dock. The charging contacts 144 can be formed from any appropriate conducting material and are electrically connected (e.g., via a printed circuit board) to a battery within the handle portion 102.

FIG. 1C is an alternative view of the device 100 in the separated state. FIG. 1C shows that the dilator portion 104 includes a dilator plate 148 that houses magnetic locating pins beneath the surface of the dilator plate 148. The dilator portion 104 includes a motor housing recess 146 in the dilator plate 148 that is sized and shaped to receive the motor housing 108. The recess 146 includes a notch 146a sized and shaped to receive the connecting tab 140 of the handle portion 102.

The dilator portion 104 includes one or more locating pins 150 extending away from the dilator plate 148. The locating pins 150 are sized and shaped to fit into the locating recesses 136 within the faceplate 132 of the handle portion 102. The locating pins 150 can be used to prevent an incorrect orientation of the dilator portion 104 to the handle portion 102 when the two parts 102 and 104 are put together.

FIGS. 1D and 1E illustrate the connection mechanism for connecting the dilator portion 104 to the handle portion 102. FIG. 1D is an orthographic (bottom) view of the dilator portion 104. FIG. 1E is an orthographic (facing the faceplate 132) view of the handle portion 102.

Magnets underneath the faceplate 132 of the handle portion 102 are matched to magnets under the dilator plate 148. The magnets are sized to produce a sufficient magnetic strength to hold the dilator portion 104 to the handle portion 102 during pelvic floor physical therapy. Some of the other features of the connection mechanism are used to ensure a correct orientation between the handle portion 102 and the dilator portion 104, i.e., an orientation the provides a sufficiently strong magnetic connection for pelvic floor physical therapy and that allows vibrations from the handle portion 102 to be felt in the dilator portion 104.

For example, the locating recesses 136 can each be teardrop shaped. For each of the locating recesses 136, a corresponding locator pin of the locator pins 150 can fit easily into a wide end of the teardrop and snugly into a narrow end of the teardrop. The connecting tab 140 of the handle portion 102 fits within the notch 146a of the recess 146 in the dilator plate 148 when the locator pins 150 are aligned with the locating recesses 136.

In this manner, a user can connect the dilator portion 104 to the handle portion 102 by pressing the dilator portion 104 against the handle portion 102 and rotating one or the other (or both) until pushing the locator pins 150 into the wide ends of the locating recesses 136. Then, the user can further rotate so that the locating pins 150 slide into the narrow ends of the locating recesses 136 and the magnets then hold the two portions 102 and 104 together securely. While rotating, the connecting tab 140 rotates within the notch 146a of the recess 146 that receives the motor housing 108.

FIG. 1F is a cross-sectional view of one example internal configuration of the device 100. The dilator portion 104 is depicted in cross section, showing the internal components and/or hardware including the motor housing 108 for a vibration motor 106 (e.g., eccentric rotating mass (ERM) vibration motor) on the right towards a tip 110 of the dilator portion 104. In some embodiments, handle portion 102 and dilator attachment portion 104 can be coupled via a connection mechanism 112.

In some embodiments, the dilator portion 104 may comprise different materials, e.g., an outer silicone layer 114 and an internal plastic material 116 (which may be primarily hollow). For example, the use of different materials and different portions can be important for balancing the weight of the device 100, which can improve device ergonomics. Notably, in some embodiments, the dilator portion 104 may not be solid silicone, but rather may include a substantially hollow inner core that helps reduce the weight of the dilator portion 104.

As shown in FIG. 1F, the device 100 may include an example connection mechanism 112 that includes a ball-nose spring plunger 118 and a plurality of pins 120 (e.g., magnetic or metallic pins, spring-loaded or pogo pins, magnetic pogo pins, etc.). For example, the spring plunger 118 and the magnetic pins 120 can be used to affix the dilator portion 104 to the handle portion 102. Notably, in some embodiments, the spring plunger 118 and the magnetic pins 120 may collectively prevent the dilator portion 104 from rotating freely against the handle portion 102. In such embodiments, this design feature can ensure that users are only able to attach the dilator portion 104 and the handle portion 102 in the correct orientation (of connection).

As shown in FIG. 1F, the ball-nose spring plunger 118 may be depicted as a small light gray circle. Notably, in some embodiments, the spring plunger 118 may include a small metallic or plastic sphere on top of a spring and can cause an audible ‘click’ to be heard when engaged, thereby notifying the user that the dilator portion 104 has been correctly and adequately attached to the handle portion 102. In some embodiments, the pins 120 may be spring-loaded or pogo pins.

In some embodiments, the pins 120 may serve as a mechanism through which electrical power is passed from the handle portion 102 (which houses a battery component) to the dilator portion 104. In some embodiments, the pins 120 may facilitate the charging of a power supply (e.g., a battery) in the handle portion 102 (e.g., when the handle portion 102 is placed or situated on a charger device or is connected to a charger device). In some embodiments, the pins 120 may be made from an electrically conductive material (e.g., metallic material) and reside in a cylinder component (e.g., a distal portion of connection mechanism) that comprises at least a substantially magnetic material. In such embodiments, the pins 120 are adapted to transfer electrical power from the handle portion 102 to the dilator portion 104.

FIG. 1G is a cross-sectional view of the device 100 in a configuration where the vibration motor 106 is located inside the handle portion 102 instead of the dilator portion 104. FIG. 1G shows one or more locating magnets 158 behind the dilator plate 148. FIG. 1G shows an example location for a battery 152 (or other appropriate energy storage device) in the wider spherical section of the handle portion 152. In this example, a printed circuit board 154 can connect the battery 152 to the motor 106, the indicator light 134, and the charging contacts 144 (when connected to a charging dock). In general, any appropriate electrical structure may be used to couple the battery 152 to electrical components of the device 100.

FIG. 1H is a cross-sectional view of the handle 102 showing the vibration motor 106 within the motor housing 108. FIG. 1H also shows locating magnets 156, which are located behind the faceplate 132. FIG. 1I is a cross-section view of the handle portion 102 and the dilator portion 104 connected via the locating pins and the locating magnets.

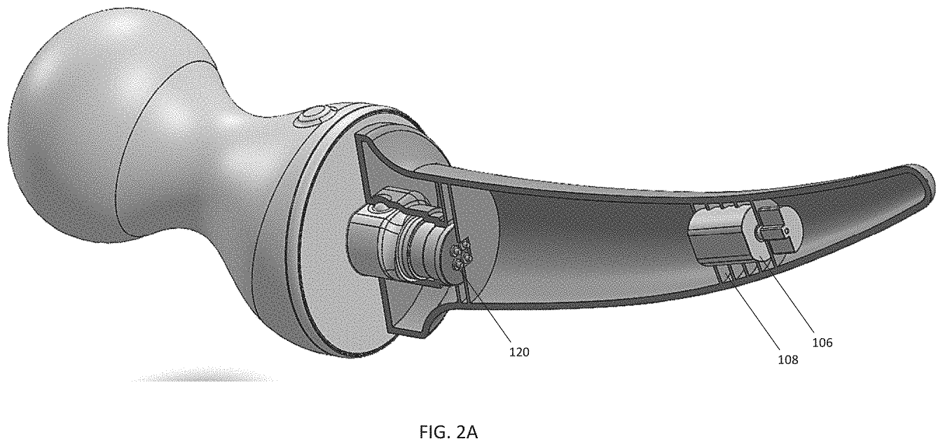

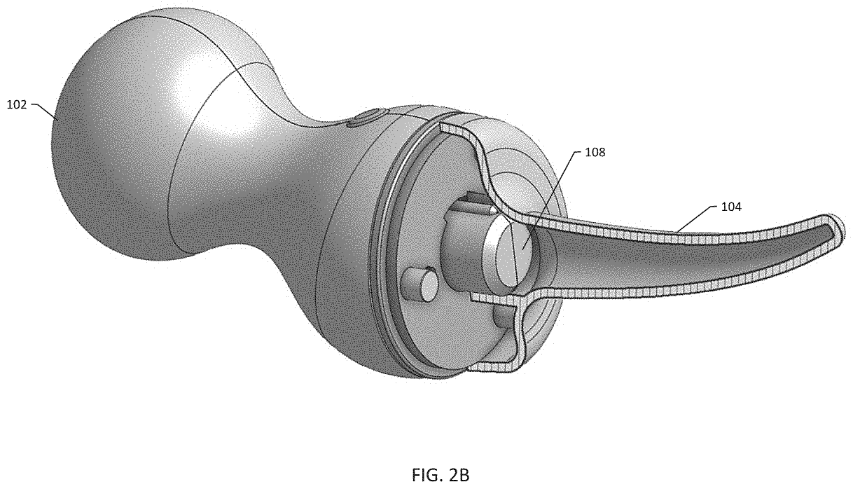

FIG. 2A is a cross-sectional view of the device 100 in a configuration where the vibration motor 106 is located in the dilator portion 104. FIG. 2A depicts that there are multiple connecting pins 120 in the handle portion 102. Further, FIG. 2A depicts a view of the stabilizing motor housing 108 surrounding the vibration motor 106. While FIG. 2A depicts four connecting pins 120 in handle portion 102, it will be appreciated that other embodiments may utilize more or less than four connecting pins 120.

FIG. 2B is a side view of the device 100 including a cross-sectional view of the dilator portion 104. In this example, the motor housing 108 is a part of the handle portion 102 and nested within a recess of the dilator portion 104.

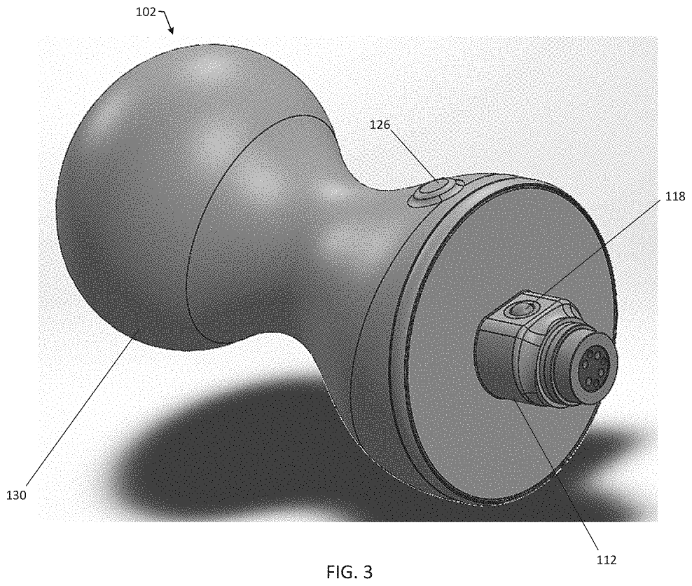

FIG. 3 is a side view of the handle portion 102 of the device 100. The handle portion 102 includes an example connection mechanism 112 on the right side of FIG. 3 and an ergonomically shaped curved handle portion 130 on the left side of FIG. 3.

In some embodiments, the handle portion 102 may include (e.g., similar to the dilator portion 104) a thermoplastic shell material. In some embodiments, the handle portion 102 may be overmolded with thermoplastic polyurethane (TPU), a liquid silicone rubber (LSR), or a rubber-like material. In some embodiments, the shell of the handle portion 102 can be configured to house a power supply, charging indication light-emitting diodes (LEDs), activation button 126, printed circuit boards (PCBs), the vibration motor 106, and/or added weight for ergonomic purposes (e.g., even or balanced weight distribution in the handle portion 102).

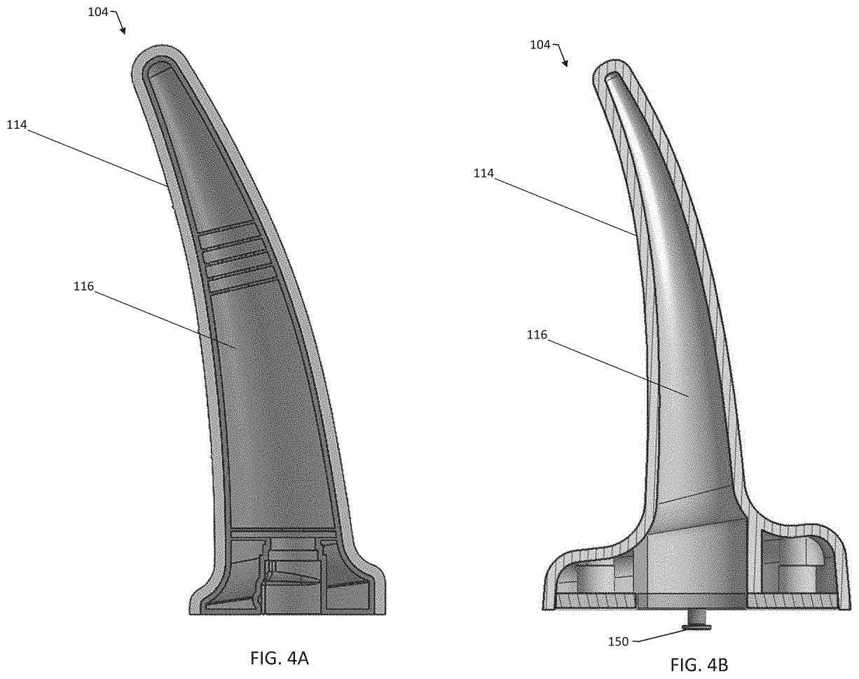

FIG. 4A is a cross-sectional view of the dilator portion 104 of the device 100. For example, the dilator portion 104 as depicted in FIG. 4 demonstrates the curve of the device, the components (e.g., a hollow hard plastic core 116 with a surrounding silicone material 114). FIG. 4B is a cross-sectional view of the dilator portion 104 in a configuration with one or more locator pins 150. In both of the examples shown in FIGS. 4A and 4B, the dilator portion 104 generally follows a gently-sloping parabolic curve.

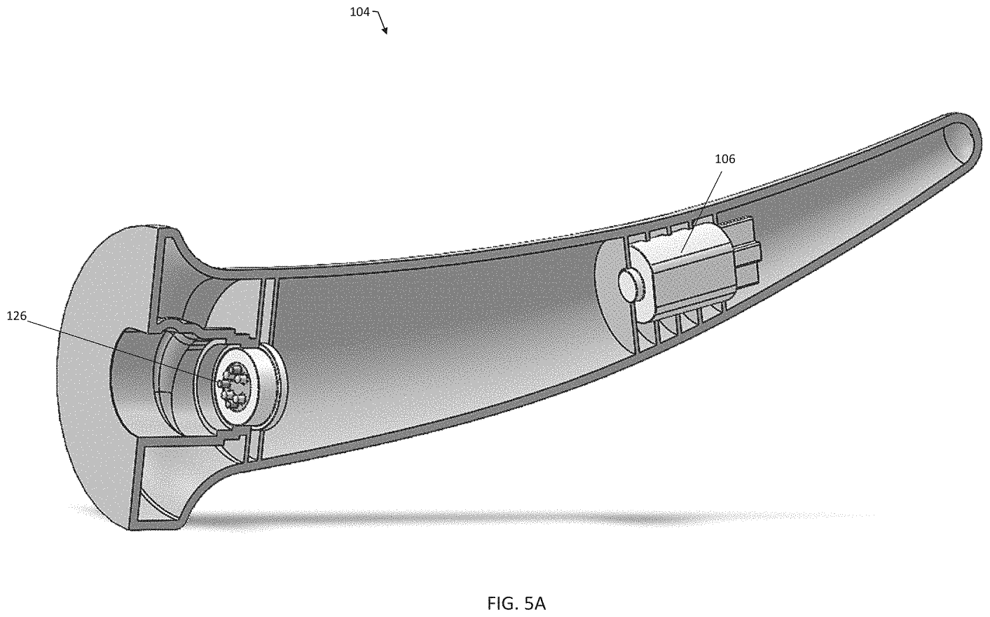

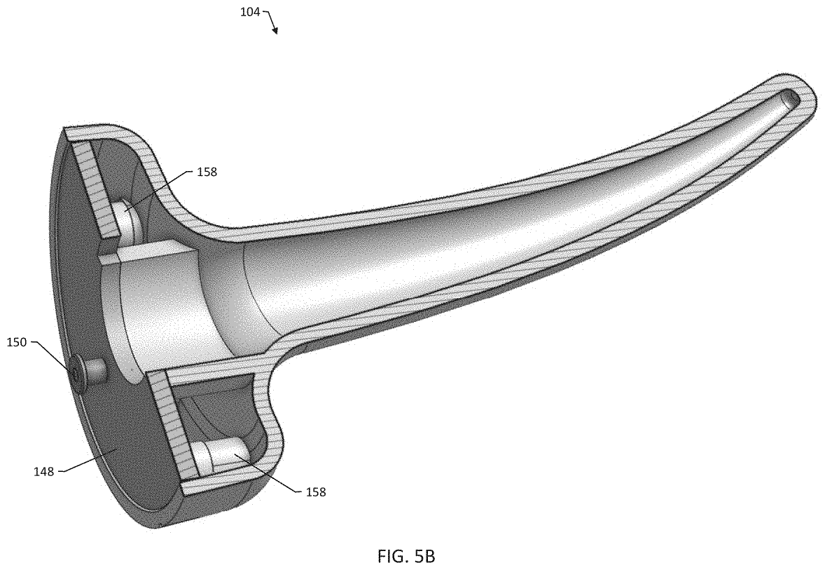

FIG. 5A is a cross-sectional view of the dilator portion 104 of the device 100. In this example, the pins 120 and the vibration motor 106 are shown within the housing of the dilator portion 104. In some embodiments, the pins 120 may vary in number and may be configured to interlock (e.g., via magnetic coupling) with a corresponding number of pins on the handle portion 102. In some embodiments, the handle portion 102 and the dilator portion 104 may each have male or female (or both) pins. In some embodiments, the pins 120 may transmit electric energy from the battery unit situated in handle portion 102 to the vibration motor 106 in the dilator portion 104 while also magnetically connecting the handle portion 102 and the dilator portion 104 to each other. In some embodiments, the vibration motor 106 can be configured to provide variable vibrational energy directed to vaginal tissue and may be usable for relaxing the tissue and promoting blood flow, which in turn can reduce pain and dyspareunia.

FIG. 5B is a cross-sectional view of the dilator portion 104 in a configuration with one or more locator pins 150 and where the dilator portion 104 lacks the vibration motor 106. FIG. 5B also shows that the dilator portion 104 can include one or more locating magnets 158 behind the dilator plate 148. The locating magnets 158 can be situated across from corresponding locating magnets in the handle portion 102 when the two portions are connected together.

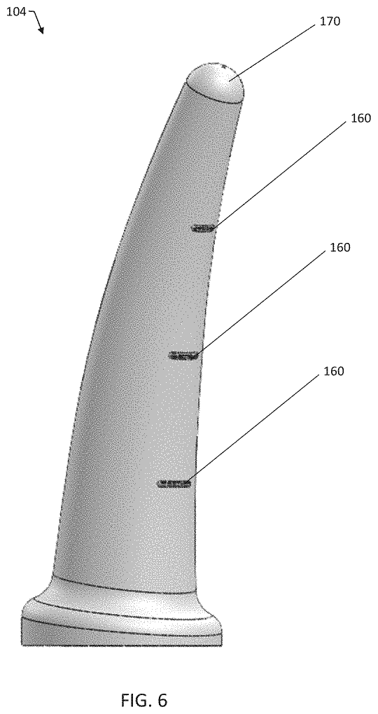

FIG. 6 is a side view of the exterior of the dilator portion 104 of the device 100. The dilator portion 104 is characterized as having a gradual decrease in circumference starting from the base (which attaches to the handle portion) which is shown at the bottom on the FIG. 6 towards the tip 170 which is rounded. The dilator portion 104 also includes a plurality of tactile gradation markings 160 on the silicone, which can be utilized by a patient user to gauge the depth of insertion. For example, gradation markings 160 may be shallow bumps or divots on the exterior that serve to tactically inform the user how deep the dilator portion 104 has been inserted into the body. It should also be noted that the base of the dilator portion 104 is not flat, and thereby contributes to the overall curve of the dilator portion.

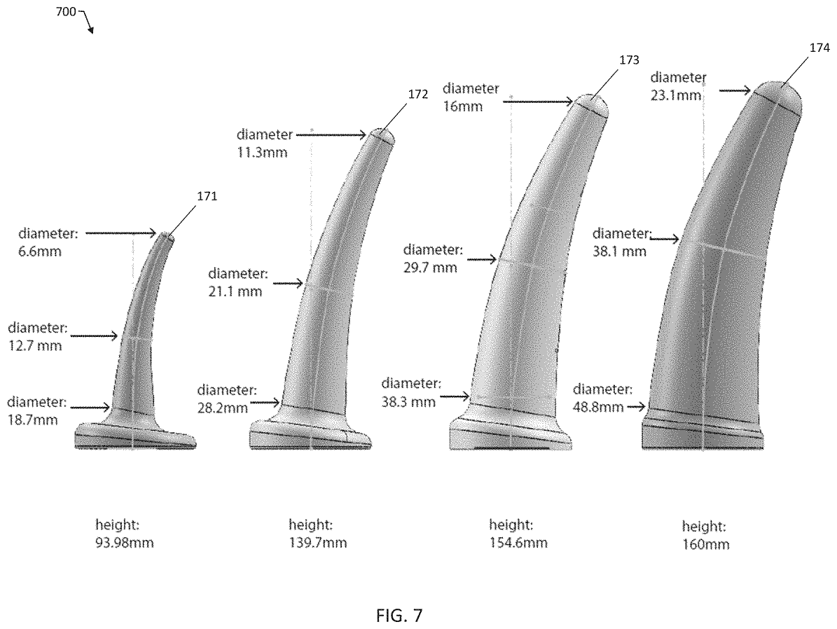

FIG. 7 is a line-up view of a plurality of different dilator attachment portions 171-174 of the device 100. For example, FIG. 7 shows four example dilator attachment portions 171-174 that range in size (e.g., again with the exterior demonstrated). In some embodiments, each of the dilator attachment portions 171-174 may have a different height, diameter(s), gradation, and/or curvature. For example, the dilator attachment portions 171-174 may graduate in size in a manner that reduces the number of dilators necessary to change size from the smallest circumference to the largest circumference. In this example, the gradual increase in dilator attachment portion size can allow a patient to gradually expand both the depth and width dimensions of their vagina as part of dilation therapy. Notably, in some embodiments, the diameter of a tip (e.g., the highest depicted diameter for a respective dilator attachment portion) of a larger size dilator (e.g., dilator attachment portion 174) is approximately (or slightly smaller than, e.g., about 75% or more) the middle diameter (e.g., the middle depicted diameter for a respective dilator attachment portion) of the next smaller size dilator attachment portion (e.g., dilator attachment portion 173. In such embodiments, some size and/or dimension overlap between two or more of the dilator attachment portions 171-174 can facilitate the ability of the user to work on either depth dilation or width dilation which can involve using multiple dilator sizes.

Moreover, the curvature of each of the dilator attachment portions 171-174 is demonstrated in FIG. 7. In some embodiments, the curvature of the dilator attachment portions 171-174 can permit or facilitate therapy that is directed laterally to the pelvic floor (as opposed to simple dilation). Examples of laterally directed therapies include myofascial release or trigger point massage, which are therapies that have been shown to reduce pain and dyspareunia. Notably, dimensions illustrated in FIG. 7 have been created and altered based upon a significant volume of feedback from device users and clinicians.

In some examples, the handle portion 102 is configured to detect which of the dilator attachment portions 171-174 is attached and adjust control of the vibration motor 106 to accommodate for mass change. For example, the handle portion 102 can be configured to increase the power supplied to the vibration motor 106 in response to detecting a larger mass.

The handle portion 102 can be configured to detect which of the dilator attachment portions 171-174 is attached using any appropriate technology. For example, the handle portion can use one or more of the following types of detection:
Mechanical detection: The handle portion 102 could use physical mechanisms to detect the attachment. For example, each attachment could have a different shape or size, and the handle portion 102 could have corresponding slots or connectors that fit only with that attachment. When the attachment is connected to the handle portion 102, the physical fit could be detected and used to determine which attachment is in use.
Electrical detection: Each attachment could have a unique electrical signature, such as a specific resistance or capacitance. The handle portion 102 could have sensors or circuits that detect these signatures and use them to identify which attachment is attached.
Radio frequency identification (RFID): Each attachment could have an RFID tag, which contains a unique identifier. The handle portion 102 could have an RFID reader that detects the tag and uses the identifier to determine which attachment is attached.
Magnetic detection: Each attachment could have a small magnet embedded in it, and the handle portion 102 could have magnetic sensors that detect the presence and position of the magnet. The pattern of magnets could be used to identify which attachment is attached.

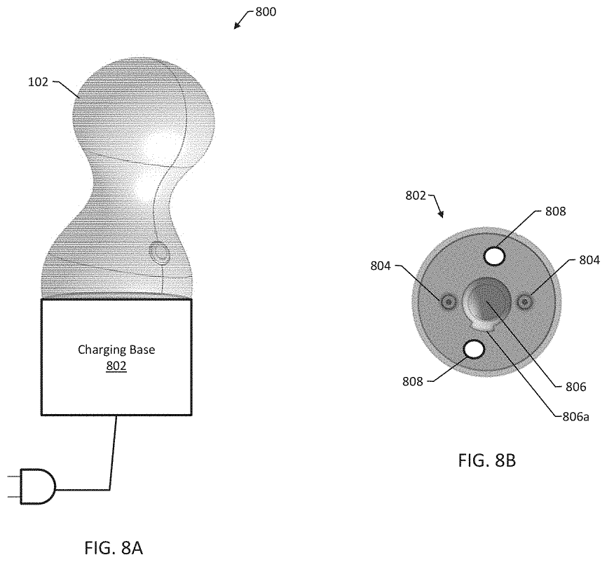

FIG. 8A is a block diagram of an example system 800 for charging the handle portion 102 of the device 100. The handle portion 102 is electrically coupled to a charging base 802 which supplies power to a battery within the handle portion 102, e.g., by way of electrical contacts on the handle portion 102. The system for charging the handle portion 102 can be useful, e.g., for allowing inconspicuous charging of the device 100. Inconspicuous charging can, in turn, encourage compliance with certain treatment programs by allowing the device 100 to remain in view.

FIG. 8B is a top view of an example configuration of the charging base 802. The charging base 802 can include one or more locator pins 804 situated to engage locator recesses on the handle portion 102. The charging base 802 can include a recess 806 sized and shaped to receive the motor housing 108 on the handle portion 102. The recess 806 can include a notch 806a sized and shaped to receive a connection tab on the handle portion 102. In this manner, the handle portion 102 can be connected to the charging base 802 in the same way that it is connected to the dilator portion 104, i.e., by pushing and rotating until locked into place. The charging base 802 also includes one or more electrical contacts 808 situated to make electrical contact with the electrical contacts on the handle portion 102.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A device comprising:
a handle portion comprising:
a curved handle portion;
a battery within the handle portion;
a motor housing including a vibration motor coupled to the battery;
an activation button configured to activate the vibration motor; and
a faceplate and one or more locating recesses within the faceplate; and
an intravaginal dilator portion comprising:
a dilator plate and a motor housing recess configured to receive the motor housing when the intravaginal dilator portion is attached to the handle portion;
one or more locating pins configured to fit within the locating recesses of the handle portion when the intravaginal dilator portion is attached to the handle portion.

2. The device of claim 1, wherein the handle portion comprises a connecting tab, and wherein the motor housing recess of the intravaginal dilator portion comprises a notch configured to receive the connecting tab when the intravaginal dilator portion is attached to the handle portion.

3. The device of claim 1, wherein the handle portion comprises one or more first magnets and the intravaginal dilator portion comprises one or more second magnets, and wherein the first magnets and the second magnets are placed to magnetically couple the intravaginal dilator portion to the handle portion when the intravaginal dilator portion is attached to the handle portion.

4. The device of claim 1, wherein the handle portion comprises a printed circuit board configured to electrically connect the battery, the vibration motor, and the activation button.

5. The device of claim 1, wherein the intravaginal dilator portion is tapered in size and follows a parabolic curve from a proximal end to a distal end.

6. The device of claim 1, comprising a second intravaginal dilator portion tapered in size, wherein a non-distal end portion of the second intravaginal dilator portion has approximately the same circumference and/or a width as a near-distal end portion of the intravaginal dilator portion and a near-distal end portion of the second intravaginal dilator has a larger circumference and/or a width as the near-distal end portion of the intravaginal dilator portion.

7. The device of claim 1, wherein a surface of the intravaginal dilator portion includes gradation depth markings.

8. The device of claim 7, wherein the gradation depth markings are tactile, visual, and/or usable for accurately gauging a depth of insertion and circumference change of the dilator attachment portion.

9. The device of claim 7, wherein the gradation depth markings indicate an approximate depth of insertion into a body.

10. The device of claim 1, wherein the handle portion includes a charging indicator light.

11. The device of claim 1, wherein the handle portion is configured to detect a size of the intravaginal dilator and to adjust motor control of the vibration motor based on the size of the intravaginal dilator.

12. A system comprising:
a handle portion comprising:
a battery within the handle portion;
a motor housing including a vibration motor coupled to the battery;
one or more handle charging contacts coupled to the battery; and
an activation button configured to activate the vibration motor;
an intravaginal dilator portion comprising a motor housing recess configured to receive the motor housing when the intravaginal dilator portion is attached to the handle portion; and
a charging base comprising one or more base electrical contacts configured to contact the handle charging contacts when the handle portion is attached to the charging base.

13. The system of claim 12, wherein the handle portion comprises a faceplate and one or more locating recesses within the faceplate, and wherein the intravaginal dilator portion comprises one or more locating pins configured to fit within the locating recesses of the handle portion when the intravaginal dilator portion is attached to the handle portion.

14. The system of claim 13, wherein the charging base comprises one or more charging base locating pins configured to fit within the locating recesses of the handle portion when the handle portion is attached to the charging base.

15. The system of claim 12, wherein the handle portion comprises a connecting tab, and wherein the motor housing recess of the intravaginal dilator portion comprises a notch configured to receive the connecting tab when the intravaginal dilator portion is attached to the handle portion.

16. The system of claim 15, wherein the charging base comprises a charging base motor housing recess configured to receive the motor housing and a charging base notch configured to receive the connecting tab when the handle portion is attached to the charging base.

17. A method comprising:
pressing together handle portion of a device and an intravaginal dilator portion of the device and rotating until one or more locating pins of the intravaginal dilator fit within one or more locating recesses of the handle portion;
further rotating to connect the handle portion of the device to the intravaginal dilator portion; and
pressing an activation button on the handle portion of the device and activating a vibration motor within the handle portion of the device.

18. The method of claim 17, wherein the handle portion comprises one or more first magnets and the intravaginal dilator portion comprises one or more second magnets, and wherein further rotating to connect the handle portion of the device to the intravaginal dilator portion comprises further rotating to move the one or more first magnets over the one or more second magnets to magnetically couple the intravaginal dilator portion to the handle portion.

19. The method of claim 17, further comprising pressing together the handle portion and a charging base and rotating until one or more charging base locating pins of the charging base fit within the one or more locating recesses of the handle portion.

20. The method of claim 17, wherein each of the locating recesses is shaped having a wide end and a narrow end, and wherein further rotating to connect the handle portion of the device to the intravaginal dilator portion comprises further rotating to move the locating pins from the wide ends of the locating recesses to the narrow ends of the locating recesses.
